(19) United States
(12) Patent Application Publication
(10) Pub. No.: US 2021/0093788 A1
(43) Pub. Date: Apr. 1, 2021

(54) ENTERAL FEEDING DEVICE AND METHOD OF USING THE SAME

(71) Applicant: F3 BIODESIGNS, LLC, San Antonio, TX (US)

(72) Inventor: Francis Fabrigas, San Antonio, TX (US)

(21) Appl. No.: 17/036,811

(22) Filed: Sep. 29, 2020

Related U.S. Application Data
(60) Provisional application No. 62/907,669, filed on Sep. 29, 2019.

(51) Int. Cl. A61M 5/31; A61M 39/10; A61M 5/315

(52) U.S. Cl. CPC A61M 5/3134; A61M 39/10; A61J 15/0092; A61M 5/31501; A61M 5/3145

(57) ABSTRACT

The present invention relates to an enteral syringe including a barrel with a first inlet, a first outlet positioned opposite the first inlet, a first side opening formed in a side of the barrel and a plunger movably mounted in the barrel where the barrel can be filled and refilled with enteral fluid without removing the plunger from the barrel.

### CROSS-REFERENCE TO RELATED APPLICATIONS

The present application claims benefit of and priority to Provisional Patent Application Ser. No. 62/907,669 filed Sep. 29, 2019 entitled ENTERAL FEEDING DEVICE AND METHOD OF USING THE SAME, the entire content of which is incorporated by reference herein.

### BACKGROUND

### Field of the Disclosure

The present invention relates generally to the field of enteral nutrition, and more particularly to an improved enteral syringe.

### Related Art

An enteral syringe is known in the art as an apparatus to administer medication, enteral fluids or other fluids to a patient via an enteral feeding tube, as well as to vent air from the patient's stomach and to check residuals of enteral fluid previously administered to the patient. When clinicians perform these tasks, particularly the administration of medications and/or enteral fluids by gravity using a presently available enteral syringe, the plunger is removed from the barrel first to allow access to the barrel cavity of the enteral syringe such that the plunger and the syringe barrel are separated. Removal of the plunger from the barrel may be necessary multiple times as the syringe is filled and refilled. Repeated removal and reinsertion of the plunger into the barrel is required when viscous enteral fluid is being administered and requires additional pushing of the plunger to expel enteral fluid out of the barrel and into the enteral tubing. The enteral syringe plunger may also be removed and separated from the barrel during the initial unclamping of the enteral tube to allow air to escape from the patient's stomach. During this process, the plunger may be separated from the barrel and may be misplaced, for example, on the patient's bed, bedside table, or other places where it may be contaminated. In such cases, the contaminated plunger may be reused and could harm the patient, especially the vulnerable ones such as the neonates, the elderly and the immunocompromised. Moreover, in conventional enteral syringes, the plunger, if separated from the enteral syringe may be misplaced and/or lost such that replacement is necessary which may add significant costs for the health care institution.

Another problem that clinicians encounter using conventional enteral syringes is clogging of the enteral tube, for example, with fragments of medications that are bigger than the inner diameter of the enteral tube. When this happens, clinicians must take extra time and effort to unclog the enteral tube or may have to completely remove and replace the enteral tube causing additional discomfort or harm to the patient and increasing costs to the institution based on the additional time and effort required of the medical staff as well as additional financial costs attributed to enteral tube replacement.

Accordingly, it would be desirable to provide an enteral syringe that allows for providing enteral fluids and/or medications to a patient that avoids these and other problems.

### SUMMARY OF THE INVENTION

It is an object of the present disclosure to provide an enteral syringe that includes a barrel with a first inlet provided on a proximal end, a first outlet provided on a distal end and a second inlet assembly, where the second inlet assembly allows access to an internal cavity of the barrel for filling and refilling of the barrel without removing a plunger.

An enteral syringe in accordance with an embodiment of the present disclosure includes a barrel having a cylindrical shape and including a central cavity; the barrel including: a first output opening positioned on a distal end thereof; a side opening formed in a side of the barrel and spaced from the first output opening; and a plunger mounted in the first barrel and movable from a retracted position where a distal end of the plunger is positioned in the barrel and above the first side opening to a compressed position wherein the distal end of the plunger is positioned between the first side opening and the first output opening.

In embodiments, the barrel includes a first inlet opening provided in a proximal end of the barrel wherein a proximal end of the plunger extends through the first inlet opening.

In embodiments, the enteral syringe includes a first external structure surrounding the side opening and extending outward from the side of the barrel.

In embodiments, the first external structure tapers from a top portion to a bottom portion.

In embodiments, the first external structure is funnel shaped.

In embodiments, the first external structure is tube shaped.

In embodiments, the first external structure includes a puncture structure including: a base; a pointed protrusion extending upward from the base; and an opening formed in the base and in fluid communication with the side opening.

In embodiments, the enteral syringe includes a filter element positioned in the side opening.

In embodiments, the enteral syringe includes a first filter element positioned in a flow path extending between the first external structure, the side opening and the central cavity.

In embodiments, the enteral syringe includes a lid attached to a top of the first external structure and movable between a first position in which the side opening is exposed and a second position in which the first side opening is covered.

In embodiments, the lid includes a hydrophobic filter.

In embodiments, the proximal end of the plunger t remains outside of the barrel and includes a flat surface configured for contact with a user's finger to aid the user in manipulating the plunger.

In embodiments, the flat surface includes a hanger opening configured to receive a protrusion to support the enteral syringe.

In embodiments, the plunger includes a distal end that moves within the barrel and includes a seal element.

In embodiments, the seal element engages an inner wall of the central cavity such that the plunger stays in place in the barrel when no force is applied by a user to the plunger.

In embodiments, the seal element is movable along the inner wall of the central cavity when the user applies pressure to the plunger.

In embodiments, the seal element is made of an elastomeric material.

In embodiments, the barrel is made of polypropylene.

In embodiments, the enteral syringe includes a seal ring extending from an inner surface of an inner wall of the central cavity to mark the retracted position of the plunger.

In embodiments, the distal end of the barrel tapers in an area surrounding the first outlet.

In embodiments, the distal end of the syringe surrounding the first outlet is structured for connection with an enteral tube.

An enteral syringe in accordance with another embodiment of the present disclosure includes a barrel having a cylindrical shape and including a central cavity; the barrel including: a first output opening positioned on a distal end thereof; a side opening formed in a side of the barrel and spaced from the first output opening; and a bulb structure mounted on a proximal end of the barrel and in fluid communication with the central cavity such that compression of the bulb structure increases pressure in the central cavity to push fluid in the central cavity toward the first outlet.

In embodiments, the enteral syringe includes a lid configured to selectively cover the side opening during operation of the bulb structure.

In embodiments, the enteral syringe includes a first external structure surrounding the side opening and extending outward from the side of the barrel.

In embodiments, the enteral syringe includes a lid attached to a top of the first external structure and movable between a first position in which the side opening is exposed and a second position in which the first side opening is covered.

### BRIEF DESCRIPTION OF THE DRAWINGS

Exemplary embodiments of the present invention will be described with references to the accompanying figures, wherein:

FIG. 1 illustrates a front view of an enteral syringe with the plunger removed from the barrel in accordance with an embodiment of the present disclosure;

FIG. 1A illustrates a front view of an enteral syringe with the plunger inserted in the barrel in accordance with another embodiment of the present disclosure;

FIG. 1B illustrates a front view of the enteral syringe of FIG. 1A including a plunger with a round shaft in accordance with another embodiment of the present disclosure;

FIG. 1C illustrates a front view of the enteral syringe of FIG. 1A including a plunger with a partially round shaft in accordance with another embodiment of the present disclosure;

FIG. 2 is illustrates a front view of the syringe of FIG. 1 with the plunger provided in a retracted (filling) position in the barrel;

FIG. 2A illustrates a cross section of the enteral syringe of FIG. 1A;

FIG. 3 illustrates a cross sectional view of the syringe of FIG. 2;

FIG. 3A illustrates a perspective view of the enteral syringe of FIG. 1A;

FIG. 4 illustrates a perspective view of an enteral syringe also showing a hanging element in accordance with an embodiment of the present disclosure;

FIG. 5 illustrates a detailed perspective view of a secondary inlet assembly of the enteral syringed of FIG. 1 showing a side opening in the barrel with a filter in accordance with an embodiment of the present disclosure;

FIG. 6 illustrates a perspective view of a syringe including a closable secondary inlet assembly in accordance with another embodiment of the present disclosure;

FIG. 6A is a perspective view of an enteral syringe including a bulb structure mounted on a proximal end of the barrel thereof in accordance with an embodiment of the present disclosure;

FIG. 6B is a more detailed view of the second inlet assembly of the enteral syringe of FIG. 6A with a lid in an open position in accordance with an embodiment of the present disclosure;

FIG. 6C is a side view of an enteral syringe including a bulb structure mounted on the proximal end of the barrel thereof in accordance with another embodiment of the present disclosure;

FIG. 7 illustrates a side perspective view of the syringe of FIG. 6 with the lid of the secondary inlet assembly in an open position showing a hydrophobic filter on the lid in accordance with an embodiment of the present disclosure;

FIG. 8 illustrates a detailed side perspective view of the syringe of FIG. 6 showing the secondary inlet assembly without a filter element between the barrel cavity and secondary inlet assembly in accordance with an embodiment of the present disclosure;

FIG. 9 illustrates a detailed side perspective view of the syringe of FIG. 6 including a filter between the barrel cavity and the secondary inlet assembly in accordance with an embodiment of the present disclosure;

FIG. 10 illustrates a detailed view of a puncture element provided in a secondary inlet assembly of the syringe of FIG. 6 in accordance with an embodiment of the present disclosure; and

FIG. 11 illustrates a bottom perspective view the syringe of FIG. 6 including the puncture element of FIG. 10.

### DETAILED DESCRIPTION OF THE EMBODIMENT

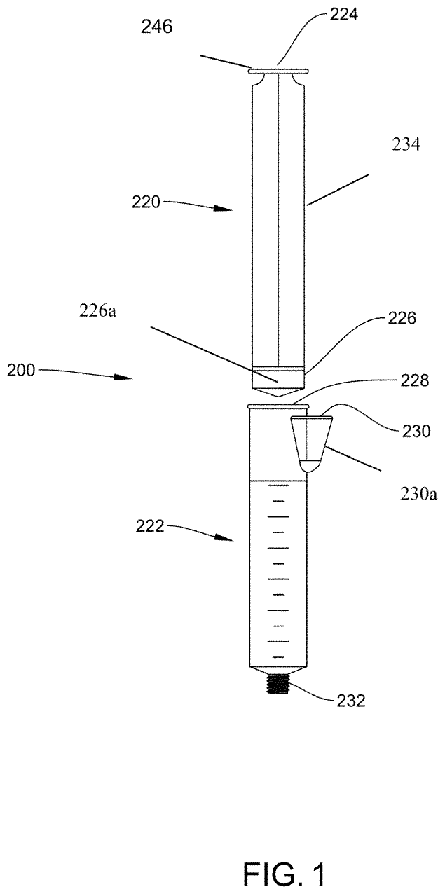

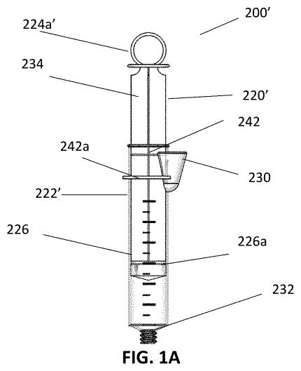

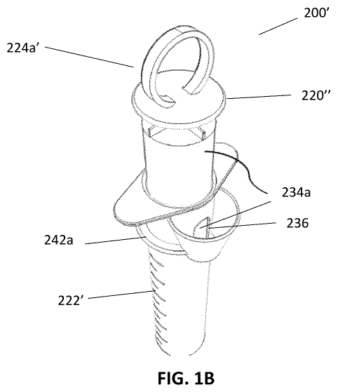

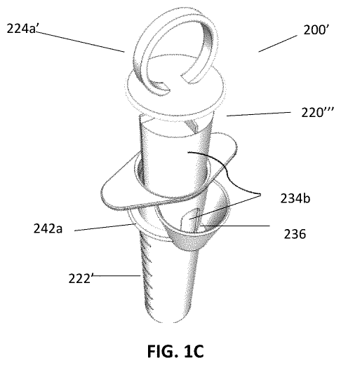

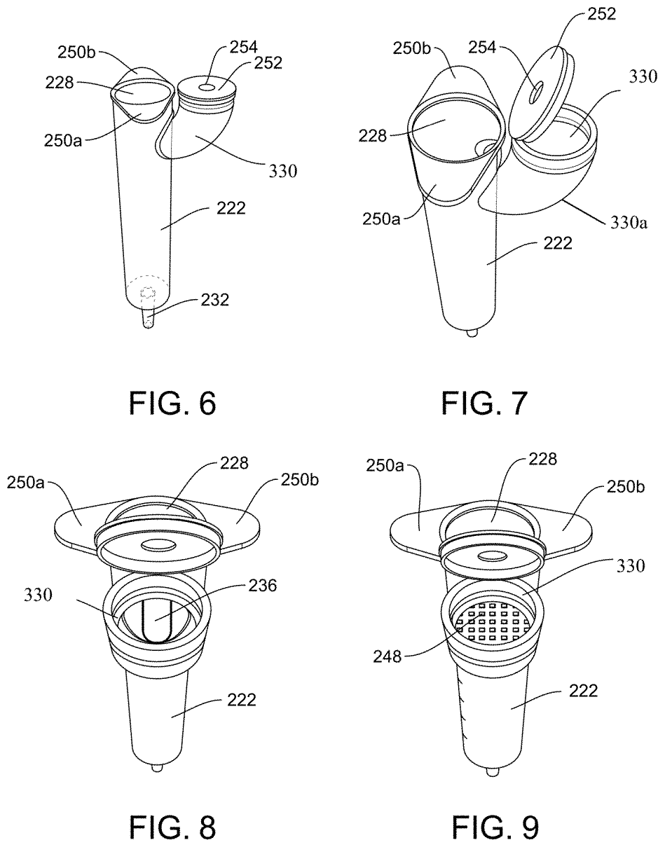

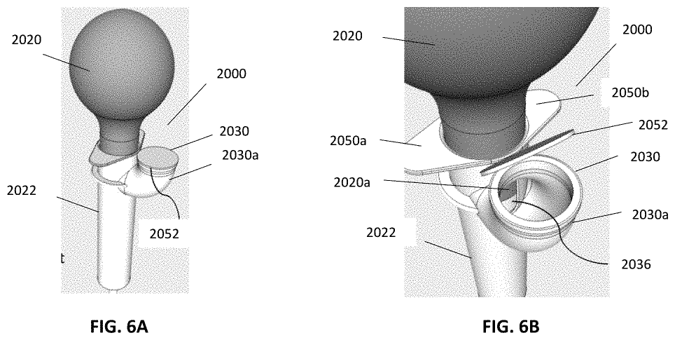

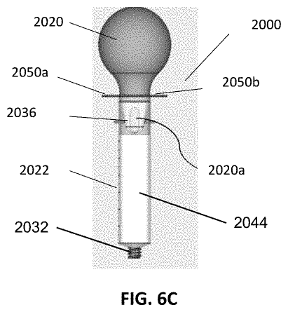

In embodiments, an enteral syringe 200 (see FIG. 1, for example) allows a user to keep a barrel 222 and plunger 220 together during filling or refilling of the enteral syringe. In embodiments, the enteral syringe 200 may have a secondary inlet assembly 230, 330 (see FIGS. 1 and 6, for example) separate from the first inlet 228 that receives the plunger 220 and permits access to the syringe barrel cavity 244 of the barrel 222 for filling, refilling or venting the syringe barrel and allows the user to perform said tasks without the need to completely remove the plunger 220 from the syringe barrel 222. In embodiments, it is desirable to provide an enteral syringe 200 that provides filtering of the enteral mixture/solution/medication to help prevent clogging of the enteral tube to reduce the risk of discomfort to the patient and minimize additional costs to institutions. In embodiments, it would also be desirable to have a syringe device 200 that is safe and easy for clinicians to use. In embodiments, it would also be desirable to have a method of administering the enteral fluid or medication that minimizes the steps taken by the clinician to administer the enteral fluids and medication.

In embodiments, the enteral syringe 200 allows for a method of administering enteral fluid or medication using the syringe that is effective and avoids the aforementioned deficiencies by allowing operation while keeping the plunger 220 and barrel 222 together during filling and refilling the cavity with enteral fluid and/or medication and while venting air from the user's stomach. In embodiments, the enteral syringe 200 may also allow for filtering enteral mixtures or solutions prior to their entering a patient's enteral tube to reduce the likelihood of clogging.

In embodiments, an enteral syringe 200 may include a novel secondary inlet assembly 230, 330. In embodiments, the enteral syringe 200 may include a barrel 222 with a primary inlet 228, a secondary inlet assembly 230, 330 and a plunger 220 wherein the secondary inlet assembly includes a filter 248 (see FIGS. 5 and 9, for example).

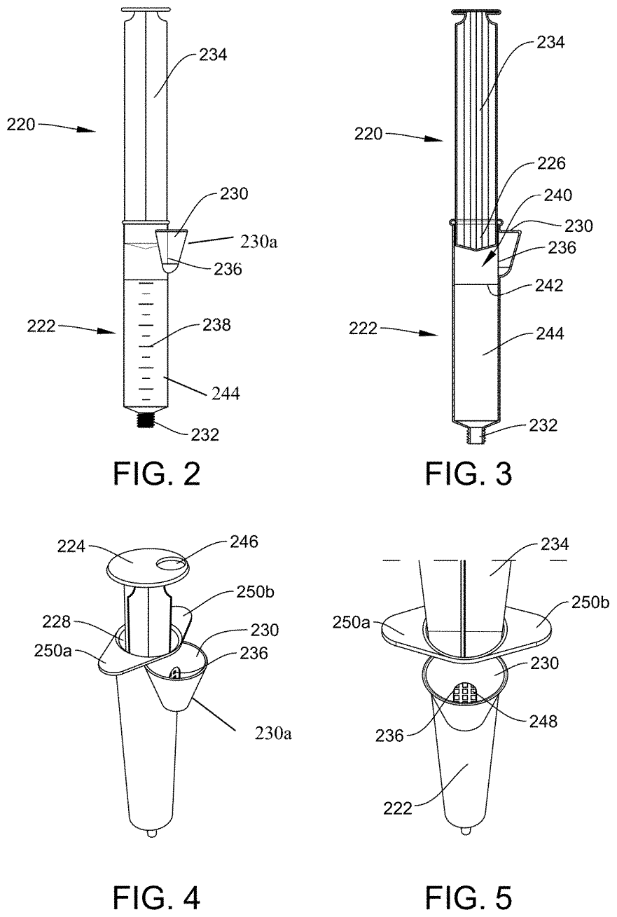

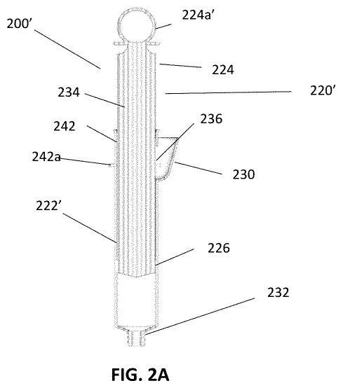

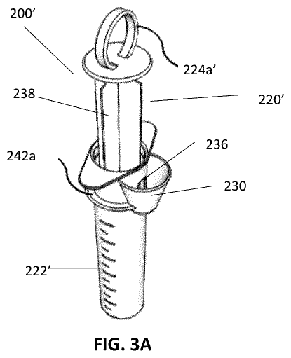

In embodiments, the barrel 222 may be intended and configured for containing the enteral nutrients and/or medications to be supplied to the patient. In embodiments, the barrel 222 may be a cylindrical tube which is generally hollow to form a cavity 244 with open ends. In embodiments, the distal opening is an outlet 232 and is configured to connect to an open end of an enteral tube. In embodiments, the proximal opening 228 on the other end of the barrel 222 may be a first, or primary, inlet that is configured to receive or otherwise accommodate the plunger 220. In embodiments, finger pads 250a, 250b (see FIG. 4, for example) may be provided adjacent to the primary (first) inlet 228 and extend outward on opposite sides of the first inlet. In embodiments, a second inlet assembly 230, 330 may be provided on the side of the barrel 222 perpendicular to the finger pads 250a, 250b. In embodiments, the second inlet assembly 230, 330 may include a funnel-like exterior structure 230a (see FIG. 1, for example). In embodiments, the second inlet assembly 230 may include a side opening 236 (see FIGS. 2, 3 and 8, for example) formed in the side of the barrel 222 that is in fluid communication with the barrel cavity 244. In embodiments, the secondary inlet assembly 230 allows alternate access to the cavity 244 such that a user has the ability to position a distal sealing end 226 (see FIG. 3, for example) of the plunger 220 in the first inlet 228 of the barrel 222 during filling, refilling or venting such that the plunger need not be removed from the barrel during operation. In embodiments, a filter 248 may be provided in or upstream from the side opening 236 in the barrel 222 to prevent the passing of mixtures or particles that may cause clogging of the enteral tube. In embodiments, different filters may be used depending on the application or on the size of the enteral tube used with the syringe 200. In embodiments, at least two different filter sizes may be used taking into consideration enteral tube size used for adults and enteral tube size used for children. In embodiments, filter size refers to spaces in the filter that allow material to pass through. In embodiments additional filter sizes may be used based on application.

In embodiments, the plunger 220 may include a proximal, retracted end 224 that remains outside of the barrel 222 and includes a flat surface 224a to aid in manipulating the plunger 220 into and out of the barrel. In embodiments, the flat surface 224a provide a surface to which a user may apply pressure to the plunger 220, for example, using a finger or otherwise. In embodiments, an X-shaped shaft 234 (see FIGS. 1-3, for example) may extend between the retracted end 224 and the sealing end 226 (see FIGS. 2-3, for example) of the plunger 220. In embodiments, the shaft 234 may be other shapes. In embodiments, the distal sealing end 226 includes a seal member 226a configured to form a seal with the interior wall surface of the barrel cavity 244 in the barrel 222.

In embodiments, the enteral syringe 200 may also include a lid 252 (see FIGS. 6-7, for example) selectively covering the secondary (second) inlet assembly 330, for example. In embodiments, the lid 252 may be removable. In embodiments, the lid 252 may be nonremovable. In embodiments, the lid 252 may be movable between an open position (see FIG. 7, for example) in which the side opening 236 is exposed and a closed position (see FIG. 6, for example) in which the side opening 236 is covered and concealed. In embodiments, the lid 252 may include a hydrophobic filter 254 (see FIG. 6, for example). In embodiments, the second inlet assembly 330 may not include or be connected to a filter. In embodiments, the second inlet assembly may be coupled or uncoupled from the barrel 222. In embodiments, the second inlet assembly 230, 330 may include a side opening 236 formed in any desired side of the barrel 222. In embodiments, the retracted end 224 of the plunger 220 may include a hanger element 246 (see FIG. 4, for example) configured to allow the syringe 200 to hang from a hook or protrusion when not in use. In embodiments, the location of the secondary inlet assembly 230, 330 may be at any desired position on the side if the barrel 222. In embodiments, the second inlet assembly 230, 330 may include a tube-like exterior structure 330a (see FIGS. 6-7, for example). In embodiments, the exterior structure 330a may be removably coupled to the barrel 222.

In embodiments, the enteral syringe 200 of the present disclosure is unique when compared to conventional syringes and provides at least the following advantages: (1) a secondary inlet assembly 230, 330 that allows for filling, refilling or venting without removal of the plunger from the barrel and (2) filtering of the enteral liquid that goes into the syringe barrel 222 via the second inlet assembly 230, 330 and eventually into the enteral tubing. In embodiments, a method of providing enteral fluid using the enteral syringe 200 of the present disclosure is unique in that it: (1) allows the user to keep the barrel 222 and plunger 220 together during filling, refilling or venting; (2) improves efficiency by eliminating steps of removing the plunger from the syringe barrel every time there is a need to access the enteral syringe barrel; and (3) helps reduce the risk of plunger contamination since the plunger remains in the barrel.

The following is a more detailed and specific description made with reference to the accompanying drawings. The drawings and specific descriptions of the drawings, as well as any specific or alternative embodiments discussed, are intended to be read in conjunction with the entirety of this disclosure.

FIG. 1 illustrates an exemplary illustration of an embodiment of an enteral syringe 200 in accordance with an embodiment of the present disclosure including a plunger 220 that is illustrated as being separated from the barrel 222. In embodiments, the plunger 220 may include a proximal retracted end 224 that remains outside of the barrel 222 and includes a thumb surface 224a provided to aid in manipulation of the plunger 220 within the barrel 222. In embodiments, the retracted end 224 may be connected via a shaft 234 to the sealing end. The thumb surface 224a may be a flat surface to which the user may apply pressure to the plunger 220, which is mounted in the barrel 222 and remains in the barrel during operation of the syringe 200. In embodiments, the distal sealing end 226 may include an elastomeric seal 226a sized and configured to tightly engage the inner wall of the barrel cavity 244. In embodiments, the seal 226a frictionally engages the inner wall of the cavity 244 of the barrel 222 so that the plunger 220 remains in a particular position within the syringe cavity 244 without user manipulation. In embodiments, this feature is particularly beneficial when positioning the sealing end 226 of the plunger 220 in the first inlet 228 during filling/refilling and/or venting to keep the plunger in place. In embodiments, the plunger 220 is movable from a retracted position (see FIGS. 2-3, for example) where the sealing end 226 is mounted in the first inlet 228 to a compressed position where the sealing end 226 slides downward in the cavity 244 to expel the enteral fluid out of the outlet 232, which may be connected to the enteral tube (not shown). FIGS. 1A, 2A and 3A illustrate an exemplary plunger 220′ in a partially compressed position with the sealing end 226 positioned below the side opening 236.

FIG. 1 illustrates an exemplary illustration of an embodiment of an enteral syringe 200 including a plunger 220 that is illustrated as separated from the barrel 222, however, such separation is not necessary during operation of the syringe 200. In embodiments, the plunger 220 may include the retracted end 224 with the thumb surface 224a provided to aid in manipulation of the plunger 220 within the barrel 222. In embodiments, the retracted end 224 may be connected via an X-shaped shaft 234 to the sealing end 226. In embodiments, the shaft 234 may be any other suitable shape. In embodiments, the sealing end 226 may include an elastomeric seal 226a sized and configured to tightly engage the inner wall of the barrel cavity 244. In embodiments, the seal 226a frictionally engages the inner wall of the barrel cavity 244 so that the plunger 220 remains in a particular position within the syringe cavity 244 without user manipulation. That is, in embodiments, the plunger 220 will remain in position with respect to the barrel 222 unless and until force is applied by the user. In embodiments, this feature is particularly beneficial when positioning the sealing end 226 of the plunger 220 in the first inlet 228 during filling/refilling and/or venting such that the plunger stays connected and in place while filling, refilling and/or venting takes place.

In embodiments, the body of the syringe (barrel) 222 may be a cylindrical tube that is generally hollow and elongated including an internal barrel cavity 244. In embodiments, the barrel 222 is open on both ends. In embodiments, the primary (first) inlet 228 is provided on a proximal end of the barrel 222, and an outlet 232 is provided on the distal end of the barrel. In embodiments, a second inlet assembly 230 may be provided spaced from the primary (first) inlet 228. In embodiments, the second inlet assembly 230 may include a funnel-like exterior structure 230a and a side opening 236 formed in the side of the barrel 222 and in fluid communication with the barrel cavity 244. In the retracted position, the sealing end 226 of the plunger 220 is positioned above the side opening 236 and rests in the mouth of primary inlet 228 of the barrel 222, while access to the cavity 244 is provided via the side opening 236 to allow for filling, refilling and or venting via the cavity 244 without the need to remove the plunger 220 from the barrel 222. In embodiments, a filter 248 may be disposed in the side opening 236 of the barrel 222 to help prevent large particles from entering the barrel cavity 244 and passing through the outlet 232 into the enteral tube (not shown) where they may cause clogging. In embodiments, the distal opening (first outlet) 232 may be configured to connect to an EnFit® compatible outlet which is known in the art or any other suitable connection to an enteral tube or other tube.

FIG. 2 is an illustration of the enteral syringe 200 showing the sealing end 226 of the plunger 220 engaged in the primary inlet 228 of the barrel 222 in a retracted position during filling or refilling of the cavity 244 and/or during venting of air from the stomach.

FIG. 3 is a cross section of the enteral syringe 200 of FIG. 2 illustrating the position of the sealing end 226 of the plunger 220 in the primary inlet 228 of the barrel 222 with the arrow 240 showing flow of enteral fluid from the secondary inlet assembly 230 into the barrel cavity 244 through the side opening 236 of the barrel 222 during filling or refilling of the syringe. As can be seen in FIG. 3, with the sealing end 226 of the plunger 220 in the retracted position, the user can vent air from the patient's stomach via the enteral tube through the cavity 244 and the side opening 236 and fill or refill the cavity 244 with enteral fluid by pouring the fluid into the secondary inlet assembly 230 where the fluid will pass from the secondary inlet assembly 230 through the side opening 236 and into the barrel cavity 244 as shown by the arrow 240. In embodiments, the enteral syringe 200 avoids the need for complete separation of the plunger 220 and the barrel 222 since the syringe may be filled and refilled and stomach air may be vented without removing the plunger 220. In embodiments, a circular ring 242 with an inner diameter slightly smaller than the inner diameter of the barrel is provided to help the user determine the position of the sealing head 226a relative to the side opening 236 of the barrel 222. In embodiments, the circular ring 242 may be positioned closer to the first inlet 228, if desired (see FIGS. 1A, 2A and 3A, for example).

FIGS. 1A, 2A and 3A illustrate an enteral syringe 200′ that includes a plunger 220′ with a gripping ring 224a′ provided on the retracted end 224 in accordance with an embodiment of the present disclosure. In embodiments, the gripping ring 224a′ may be configured and sized to receive a thumb, or a finger of a user to allow for application of pressure either downward, toward the outlet 232, or upward, away from the outlet, as desired. In embodiments, the barrel 222′ may include an external flange 242a that extends around outer surface of the barrel. In embodiments, the external flange 242a provides a gripping edge for the user. In embodiments, the enteral syringe 200′ may be operated by a user using one hand and the gripping ring 224a′ and external flange 242a provide secure gripping points to allow the user to more easily manipulate the plunger 220′, particularly when moving the plunger upward, away from the outlet 232. Otherwise, the enteral syringe 200 is configured and structured in much the same manner as the syringe 200.

In embodiments, the syringe 200′ may include a plunger 220″ with a circular shaft 234a provided between the retracted end 224 and the sealing end 226 as can be seen in FIG. 1B, for example. In embodiments, a plunger 220′″ may include a partially circular shaft 234b between the retracted end 224 and the sealing end 226 as can be seen in FIG. 1C. In embodiments, the circular shaft 234a or the partially circular shaft 234b may prevent the flow of liquid through the opening 236 when the plunger 220′ is in a compressed or partially compressed position such that the sealing end 226 is positioned below the opening 236. While illustrated in conjunction with the syringe 200′, it is noted that the shaft 236 in the enteral syringe 200 may also be modified in accordance with the shafts 236a or 236b, if desired.

FIG. 4 is perspective view of the enteral syringe 200 showing the thumb surface 224a with a hole 246 formed therethrough to provide a hanging implement. In embodiment, the hole 246 may be provided as a hanging element and is configured to receive a peg or hook that supports the syringe 200. Extending outwards on both sides of the primary inlet 228 are two finger pads 250a, 250b. On one side, perpendicular to the finger pads 250a, 250b, attached to the barrel 222 is a secondary inlet assembly 230 including a funnel-like external structure 230a. The secondary inlet assembly 230 includes the external structure 230a which has a funnel like configuration where a cavity of the funnel like shape is in fluid communication with the barrel cavity 244 via the side opening 236.

FIG. 5 is a close-up perspective view of the syringe 200 with plunger 222 engaged on the primary inlet 228. Also shown is the filter member 248 in the barrel opening 236 between the secondary inlet cavity and the barrel cavity 244.

FIG. 6 shows another embodiment of the enteral syringe 200 showing only the barrel 222 with a secondary inlet assembly 330 in a tubular shape configuration. Also shown is a lid 252 covering the secondary inlet assembly 330. In the center of the lid 252 is a hydrophobic filter 254 to allow air, but not fluid to escape from the stomach when used during venting. In embodiments, the outlet end 232 may have a tapered configuration.

FIG. 7 shows the secondary inlet assembly 330 with the lid 252 in an open position allowing access to the interior of the assembly 330 as well as the side opening 236.

FIG. 8 illustrates a front perspective view of the syringe 200 of FIG. 7 from the side including the side opening 236.

FIG. 9 illustrates the syringe 200 of FIG. 8 including filter member 248 mounted in the side opening 236 of the secondary inlet assembly 330. In embodiments, the filter element 248 may be provided upstream of the opening 236 to prevent larger particles from passing through the opening and entering the cavity 244 and ultimately out the outlet 232 into the enteral tube.

In use, the enteral syringe 200, 200′ may be used for administering enteral fluids to an enteral tube by gravity. In embodiments, the enteral fluids may be administered using pressure applied to the plunger 220 to move it to the compressed position as well. In embodiments, enteral fluids that are thick or colloidal are more likely to require pressure applied by the plunger 220 to push the fluid out of the outlet 232. In embodiments, the sealing end 226 of the plunger 220 is positioned in the primary inlet 228 of the barrel 222 in retracted position to allow for venting of air from the stomach through the outlet 232, the barrel cavity 244 and the side opening 236. In embodiments, the first outlet 232 may be connected to the enteral tube (not shown) to allow venting of air from the patient's stomach since the plunger 220 is positioned above the side opening 236 to allow air to pass through the cavity 244 and the side opening 236. In embodiments, the plunger 220 which is previously inserted in the barrel 222, may also be retracted, that is moved from a compressed position to the retracted position or partially toward the retracted position, to aspirate gastric contents from the patient's stomach into the cavity 244, during a residual check, for example. In embodiments, fluids that are drawn into the cavity 244 from the user's body may be drawn toward the side opening 236 and drained from the syringe 200, for example during lavage. In embodiments, such drawing of the residual may be performed to confirm proper placement of the enteral tube and/or to check for enteral fluid residual in the patient's stomach. In embodiments, residual checks may be used to determine how much fluid has remained in the user's stomach. In embodiments, a high amount of residual fluid may indicate that the fluid is not being emptied from the stomach into the intestines which may be a sign that tube feeding should be limited or stopped altogether to prevent gastric distention and/or aspiration which may be caused by fluid buildup in the patient's stomach that back up into the lungs. In embodiments, after checking the residual enteral fluid, the residual fluid may be pushed back into the stomach by applying pressure to the plunger and moving it into the compressed position. After venting and checking of residuals, in embodiments, the plunger 220 may be retracted so that the sealing end 226 is engaged on the primary inlet 228 of the barrel 222 above the side opening 236 to allow access to the cavity 244 without completely separating the plunger 220 and barrel 222 as indicated in FIGS. 2 & 3, for example. In embodiments, the syringe 200 may be briefly disconnected from the enteral tube prior to retraction to allow air to enter the cavity 244 to stop the flow of fluid back into the cavity 244. In embodiments, using the secondary inlet assembly 230, 330, the barrel cavity 244 may be filled, or refilled, with enteral fluid that drains into the enteral tube (feeding tube). In embodiments, the volume of enteral fluid to be provided may exceed that of the barrel cavity 244 such that refilling of the cavity may be necessary to complete treatment of the patient. In embodiments, refilling is more likely to be needed when the enteral fluid has a high viscosity such that extra force is needed to move it through the outlet requiring the plunger 220 to be compressed into the barrel 222, thereby blocking the side opening 236 and necessitating the plunger to be retracted to the retracted position to regain access to the barrel cavity 244 for refilling without completely separating the barrel and plunger. In embodiments, however, refilling may not be necessary during gravity enteral administration of less viscous fluid which may not require compression of the plunger 220 since fluid continuously flows and empties freely into the enteral tube without the need for the plunger to be compressed leaving the side opening 236 open for filling on a continuous basis if desired. In embodiments, filling and refilling may be repeated seamlessly until the desired amount of enteral fluid is given to the patient. In embodiments, after the desired amount of enteral fluid is provided to the patient, the enteral tube may be flushed with water provided via the syringe 200 without the need to remove and reinsert the plunger 220 in the barrel 222 which saves substantial time. Following delivery of the enteral fluid and flushing, the syringe 200 may be disconnected from the enteral tube and the enteral tube may be closed and the enteral syringe 200 cleaned per institution policy or other standards or regulations.

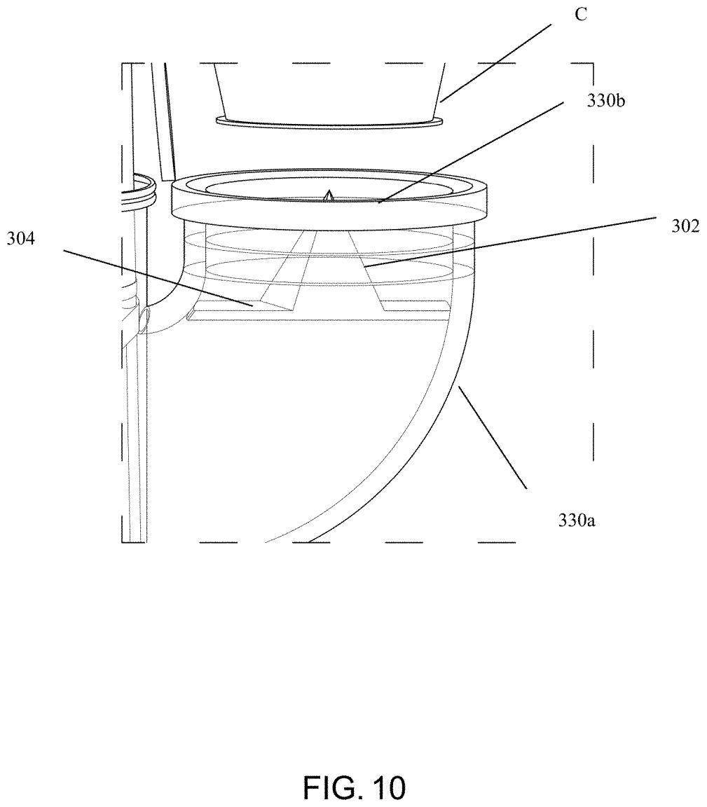

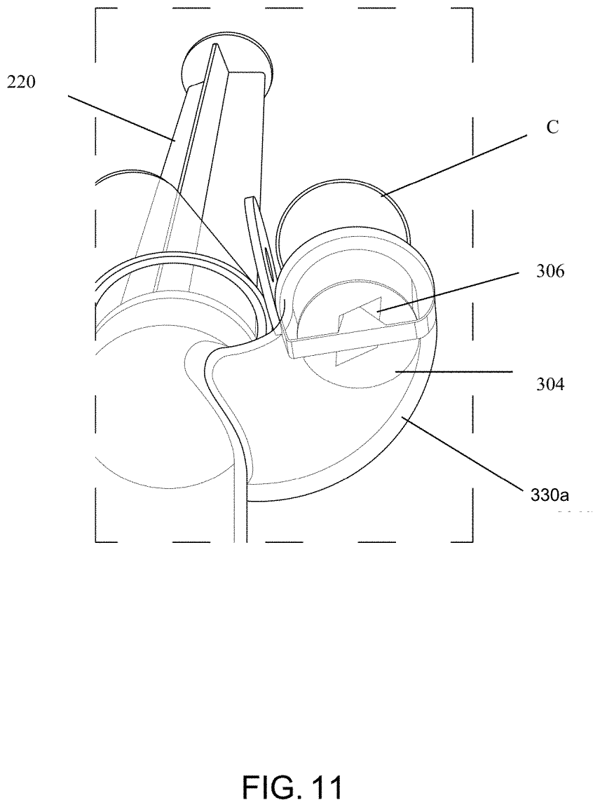

In embodiments, the secondary inlet assembly 330 may include a puncture element 330b mounted in the external structure 330a as can be seen in FIGS. 10 and 11, for example. In embodiments, puncture element 330b includes a pointed protrusion 302 extending upward from a base 304 with an opening 306 formed in the base. In embodiments, enteral medicine may be provided in pre-packaged sealed cups that are typically sealed with a flexible foil cap that may be peeled back to allow the contents to be removed. These foil caps may be difficult to pull back, especially for clinicians wearing gloves. In embodiments, the external structure 330a may be configured to accommodate such a sealed cup C such that the flexible foil cap may be punctured by the pointed protrusion 302. In embodiments, the user may then twist the cup C to widen the opening and allow the contents to flow out through the opening 306 and the side opening 236 into the cavity 244.

In embodiments, an enteral syringe 2000 (see FIGS. 6A, 6B and 6C) may include a bulb structure 2020 mounted on a proximal end of a barrel 2022 including a central cavity 2044. The barrel 2022 may include finger pads 2050a, 2050b. In embodiments, the bulb structure 2020 may be made of a flexible material. In embodiments, the bulb structure 2020 is substantially hollow and the hollow portion is in fluid communication with the cavity 2044 such that compression of the bulb structure 2020 deforms collapses the hollow area to force air into the central cavity 2044 and toward the outlet 2032. In embodiments, the force of the air entering the central cavity 2044 drives enteral fluid, or other fluid in the central cavity 2044 toward the first outlet opening 2032 provided at the distal end of the barrel 2022.

In embodiments, a side opening 2036 is formed in a side wall of the barrel 2022 and an external structure 2030a may be provided around the side opening 2036 as part of an inlet assembly 2030. In embodiments, the external structure 2030a extends from the exterior of the side wall. As indicated in FIG. 6A, the external structure 2030a has a tube shape. In embodiments, a lid 2052 may be provided on a top of the external structure 2030a to cover or seal the opening 2036 when desired as can be seen in FIG. 6A. This lid 2052 may be opened, as indicated in FIG. 6B to expose or open the opening 2036. In general, the lid 2052 should be closed when the bulb structure 2020 is manipulated by the user to apply pressure to the fluid in the cavity 2044. In embodiments, the bulb structure 2020 may also be used to draw fluid into the cavity 2044 from an enteral tube, for example. In such a case, the bulb structure 2020 may be depressed while the lid 2052 is open which allows air to escape via the opening 2036. Thereafter, the lid 2052 may be closed and the bulb structure 2020 released such that it can return to its original shape. As it expands to its original shape, suction is provided to draw fluid or air into the cavity 2044 via the outlet opening 2032 since the side opening 2036 is covered. In embodiments, a filter such as filter 248 may be provided in the side opening 2036 to prevent clogging of the enteral tube or outlet 2032.

In operation, the enteral syringe 2000 may be used to provide enteral fluid to an enteral tube by gravity in which case the lid 2052 will generally remain open to allow filing and refilling of the cavity 2044. If additional force is desirable to move material out of the outlet 2032, for example, when a viscous enteral fluid is used, lid 2052 may be closed and the bulb structure 2020 pressed and deformed by a user to force air from the bulb structure into the barrel 2044, and thus, apply force in the direction of the outlet 2032. In embodiments, pressure may be applied to the bulb structure 2020 to move it down further into the barrel 2022 such that a sidewall 2020a covers the opening 2036 such that further deformation of the bulb structure will force air into the cavity 2044 and toward the outlet 2032 as indicated in FIG. 6C. In embodiments where the bulb structure 2020 is movable into the barrel 2044 to cover the opening 2036, the lid 2052 may not be necessary.

As noted above, if suction is desired, the bulb structure 2020 may be depressed while the lid 2052 is in the open position to force air out through the opening 2036. Thereafter, the lid 2052 may be closed to seal the opening 2036 and the bulb structure 2020 may be released to allow the bulb structure 2020 to return to its original position and provide suction into the cavity 2044 through the outlet 2032.

In embodiments where the bulb structure 2020 is movable into the barrel 2022 and suction is desired, the bulb structure may be compressed without moving it down such that the sidewall 2020a does not cover the opening 2036 and air escapes the barrel 2022. Thereafter, the bulb structure may be moved down to cover the opening 2036 and the bulb structure released so that it returns to its prior shape to suck air into the barrel 2044 through the opening 2032.

In embodiments, the components of the enteral syringe 200, 200′, 2000 discussed herein may be formed from a variety of materials as desired by a user. In example embodiments, the enteral syringe 200, 200′, 2000 may be made of, or include, plastics, other polymers, glass, metals, metal alloys, resins, or any other known and suitable materials. In embodiments, the syringe body or barrel 222 may be formed from polypropylene, and the plunger 220 may be formed from the same. In embodiments, the seal 226a may be formed from an elastomeric material. In embodiments, color additives may be added to provide protection from UV light. In embodiments, colorants may be added to the syringe 200, 200′, 2000 as desired, for example, to identify certain properties/characteristics (i.e. administration path) or contents. In embodiments, the enteral syringe 200, 200′, 2000 may include external markings 238 (see FIG. 2, for example) to indicate volume capacity and remaining content levels. In embodiments the syringe 200, 200′, 2000 may include additional inlets. In embodiments, each of the additional inlets may include a filter, if desired, such as the filter 248 discussed above. In embodiments, the additional inlets may be coupled and uncoupled from the barrel 222. In embodiments, the secondary inlet assembly 230, 330 may also be provided at multiple points.

In embodiments, the syringe may be manufactured using injection molding or other suitable manufacturing techniques. In embodiments, the enteral syringe 200, 200′, 2000 may also be used in other applications such as veterinary, automotive and other industries. The syringe 200, 200′, 2000 may also be used for other applications within the medical field. In embodiments, the syringe 200, 200′ may be used for bladder irrigation, for example.

The above description is given by way of example, and not limitation. Given the above disclosure, one skilled in the art could devise variations that are within the scope and spirit of the invention disclosed herein, including various ways of fabricating the barrel, plunger or other components. Further, the various features of the embodiments disclosed herein can be used alone, or in varying combinations with each other and are not intended to be limited to the specific combination described herein. Thus, the scope of the claims is not to be limited by the illustrated embodiments.

Now that embodiments of the present invention have been shown and described in detail, various modifications and improvements thereon can become readily apparent to those skilled in the art. Accordingly, the exemplary embodiments of the present invention, as set forth above, are intended to be illustrative, not limiting. The spirit and scope of the present invention is to be construed broadly.

What is claimed is:

1. A syringe comprising:
a barrel having a cylindrical shape and including a central cavity;
the barrel comprising:
a first output opening positioned on a distal end thereof;
a side opening formed in a side of the barrel and spaced from the first output opening; and
a plunger mounted in the first barrel and movable from a retracted position where a distal end of the plunger is positioned in the barrel and above the first side opening to a compressed position wherein the distal end of the plunger is positioned between the first side opening and the first output opening.

2. The syringe of claim 1, further comprising a first inlet opening provided in a proximal end of the barrel wherein a proximal end of the plunger extends through the first inlet opening.

3. The enteral syringe of claim 1 further comprising a first external structure surrounding the first side opening and extending outward from a side of the barrel.

4. The enteral syringe of claim 3, wherein the first external structure tapers from a top portion to a bottom portion.

5. The enteral syringe of claim 4, wherein the first external structure is funnel shaped.

6. The enteral syringe of claim 3, wherein the first external structure is tube shaped.

7. The enteral syringe of claim 3, wherein the first external structure further comprises a puncture structure including:
a base;
a pointed protrusion extending upward from the base;
an opening formed in the base and in fluid communication with the first side opening.

8. The enteral syringe of claim 1, further comprising a filter element positioned in the first side opening.

9. The enteral syringe of claim 3, further comprising a filter element positioned in a flow path extending between the first external structure, the first side opening and the central cavity.

10. The enteral syringe of claim 3, further comprising a lid attached to a top of the first external structure and movable between a first position in which the first side opening is exposed and a second position in which the first side opening is covered.

11. The enteral syringe of claim 10, wherein the lid further comprises a hydrophobic filter.

12. The enteral syringe of claim 2, wherein the proximal end of the plunger remains outside of the barrel and includes a flat surface configured for contact with a user's finger to aid the user in manipulating the plunger.

13. The enteral syringe of claim 12, wherein the flat surface further comprises a hanger opening configured to receive a protrusion to support the syringe.

14. The enteral syringe of claim 12, wherein the distal end of the plunger moves within the barrel and includes a seal element.

15. The enteral syringe of claim 14, wherein the seal element engages an inner wall of the central cavity such that the plunger stays in place in the barrel when no force is applied by a user to the plunger.

16. The enteral syringe of claim 15, wherein the seal element is movable along the inner wall of the central cavity when the user applies pressure to the plunger.

17. The enteral syringe of claim 14, wherein the seal element is made of an elastomeric material.

18. The enteral syringe of claim 17, wherein the barrel is made of polypropylene.

19. The enteral syringe of claim 1, further comprising a seal ring extending from an inner surface of an inner wall of the central cavity to mark the retracted position of the plunger.

20. The enteral syringe of claim 1, wherein a distal end of the barrel tapers in an area surrounding the first outlet.

21. The enteral syringe of claim 1, wherein a distal end of the syringe surrounding the first outlet is structured for connection with an enteral tube.
